(19) United States
(12) Patent Application Publication
(10) Pub. No.: US 2012/0220905 A1
(43) Pub. Date: Aug. 30, 2012

(54) METHODS AND PROTOCOLS FOR PROVIDING ARTIFICIALLY INDUCED VIBRATIONS (AIVS) TO MAMMALIAN PATIENTS FOR THERAPEUTIC PURPOSES

(75) Inventor: Yuval Avni, Tel Aviv (IL)

(73) Assignee: ADS & B INVESTMENT FUND L.P, Herzliya Pituach (IL)

(21) Appl. No.: 13/501,175

(22) PCT Filed: Oct. 11, 2010
(86) PCT No.: PCT/IL10/00822

§ 371 Date: May 1, 2012

Related U.S. Application Data
(60) Provisional application No. 61/250,528, filed on Oct. 11, 2009.

(51) Int. Cl. A61H 1/00; A61N 7/00

(52) U.S. Cl. 601/2; 601/46

(57) ABSTRACT

The present invention discloses a method of producing therapeutic oscillations useful in treating a mammalian patient comprising steps of: obtaining an artificially induced vibrations device (AIVD) useful for a patients wellness for producing artificially induced vibrations of any of a given frequency, pressure, amplitude, volume and duration according to a protocol; said AIVD comprising: a. plurality of regulators, each of which is adapted to define an AIVD; b. mechanism for producing the protocol of said defined individual AIVs; and, c. effector adapted to introduce said protocol of produced AIVs to impinge in or on at least at least one body portion of a patient; wherein said therapeutic protocol of AIVs comprises two or more different individual time resolved AIVs.

### FIELD OF THE INVENTION

A vibrations device useful for a patients wellness for producing artificially induced vibrations of any of a given frequency, pressure, amplitude, volume and duration according to a protocol.

### BACKGROUND

There are several medical conditions which are currently treated by pharmaceutical, surgical and other therapies. There is an increasing need to find efficacious non invasive therapies. Some examples are outlined below, and the current invention is a further step in the quest to improve patient care.

PERIPHERAL VASCULAR DISEASE (PVD) is a clinical state caused by occlusion of arteries. It is therefore also known as Peripheral Arterial (Occlusive) Disease (PAD, PAOD).

Treatment includes several available measures today:
Conservative measures: smoking cessation, low-lipid diet, physical exercise, medications (for blood dilution, cholesterol chelation).
Surgical revascularization: available options include: balloon angioplasty, for solitary lesions in large arteries bypass grafting, for extended lesions and for fully or almost-fully occluded arteries. Plaque excision.
Ulcers require additional local treatment, such as off-loading, debridement, dressings.
Gangrene most often requires amputation.

Thus non invasive effective treatment for PVD is an unmet and long felt need.

### Diabetic Foot

Prolonged Diabetes Mellitus (D.M.) affects the nervous and vascular systems in multiple ways that give rise to a handful of pathologies in the patient's foot. Among many attempts to relieve these pathologies are : Negative Pressure Wound Therapy, Hyperbaric Oxygen Therapy, and Intermittent Pneumatic Compression Therapy . even after these treatments, the end stages of the disease often involve amputation, and therefore a non invasive effective treatment for diabetic and peripheral circulation pathologies of the foot is an unmet and long felt need.

### Heart

Enhanced external counterpulsation (EECP) is a non-invasive treatment that uses timed, sequential inflation of pressure cuffs on the calves, thighs and buttocks to augment diastolic pressure, decrease left ventricular afterload, and increase venous return.

Asthma and COPD (chronic obstructive pulmonary disease) are chronic illnesses requiring lifetime therapy and affect 44 million Americans. The mainstay of treatment is inhaler therapies. The delivery of medications via inhaler is problematic causing decreased efficacy and poor patient compliance. Therefore there is a constant search for improving the delivery of drugs through inhalers and positive pressure pulmonary ventilation.

### SUMMARY OF THE INVENTION

It is an object of the present invention to disclose a method of producing therapeutic oscillations useful in treating a mammalian patient comprising steps of obtaining an artificially induced vibrations device (FIG. 1) (AIVD) useful for a patients wellness for producing artificially induced vibrations of any of a given frequency, pressure, amplitude, volume and duration according to a protocol; said AIVD comprising:
a. a plurality of regulators (110), each of which is adapted to define an AIV with an individual frequency, pressure amplitude, volume and duration;
b. means or mechanism for producing the protocol (120) of said defined individual AIVs; and,
c. effector (130) adapted to introduce said protocol of produced AIVs to impinge in or on at least at least one body portion of a patient;
   wherein said therapeutic protocol of AIVs comprises two or more different individual time resolved AIVs.

It is an object of the present invention to disclose the aforementioned method wherein said AIVD is additionally provided with means for producing at least one set of predetermined humming therapeutic oscillations said humming therapeutic oscillations provided independently or contemporaneously with said series of AIVs

It is an object of the present invention to disclose the aforementioned method wherein said AIVD is additionally provided with means for massaging and/or compressing at least one body portion of a patient in a predetermined manner.

It is an object of the present invention to disclose the aforementioned method wherein said AIVs are characterized by parameters selected from a group consisting of Frequency, Pressure, amplitude, Volume and Duration.

It is an object of the present invention to disclose the aforementioned method wherein said Frequency of AIVs is selected from a group consisting of about 0.5 Hz to about 5.0 Hz, about 5.0 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, any integer multiples thereof.

It is an object of the present invention to disclose the aforementioned method wherein said protocol is dependent upon said patient's natural involuntary or voluntary functions.

It is an object of the present invention to disclose the aforementioned method wherein any of said selected Frequency is deliverable with its corresponding overtones or multiples thereof.

It is an object of the present invention to disclose the aforementioned method wherein said AIVs amplitudes are selectable on the basis of clinical efficacy.

It is an object of the present invention to disclose the aforementioned method The aforementioned AIVD wherein said volumes are selectable on the basis of clinical efficacy.

It is an object of the present invention to disclose the aforementioned method wherein said AIVD is adapted for external use on a patient's body.

It is an object of the present invention to disclose the aforementioned method wherein said AIVD is adapted for internal use on a patient's body.

It is an object of the present invention to disclose the aforementioned method wherein said AIVD is additionally provided with means for drug delivery.

It is an object of the present invention to disclose the aforementioned method wherein said therapeutic protocol comprises at least one individual AIV.

It is an object of the present invention to disclose the aforementioned method wherein said AIVD is adapted for administering said protocol to said patients respiratory system.

It is an object of the present invention to disclose the aforementioned method wherein said AND is adapted for administering said protocol to said patients circulatory system.

It is an object of the present invention to disclose the aforementioned method wherein said AIVD is adapted for administering said protocol to said patients lymphatic system.

It is an object of the present invention to disclose the aforementioned method wherein said AIVD is adapted for administering said protocol to said patient's cardiopulmonary system.

It is an object of the present invention to disclose a protocol of artificially induced vibrations (AIVs) useful for improving a patients wellness; said protocol comprising sequences of different AIVs; wherein said protocol is administered to at least one body portion of the patient; further wherein said sequences are independent of said patient's natural involuntary or voluntary functions.

It is an object of the present invention to disclose the aforementioned protocol wherein said protocol is dependent upon said patient's natural involuntary or voluntary functions.

It is an object of the present invention to disclose the aforementioned protocol wherein said AIVs are transmitted to said at least one body portion of a patient by pneumatic means.

It is an object of the present invention to disclose the aforementioned protocol wherein said AIVs are transmitted to said at least one body portion of a patient by mechanical means.

It is an object of the present invention to disclose the aforementioned protocol wherein said ATVs are transmitted to said at least one body portion of a patient by hydraulic means.

It is an object of the present invention to disclose the aforementioned protocol wherein said AIVs are transmitted to said at least one body portion of a patient by electrical means.

It is an object of the present invention to disclose the aforementioned protocol wherein said AIVs are transmitted to said at least one body portion of a patient by ultrasound means.

It is an object of the present invention to disclose the aforementioned protocol wherein said AIVs are characterised by parameters selected from a group consisting of Frequency, Pressure, amplitude, Volume and Duration.

It is an object of the present invention to disclose the aforementioned protocol wherein said Frequency of AIVs is selected from a group consisting of about 0.5 Hz to about 5.0 Hz, about 5.0 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, any integer multiples thereof.

It is an object of the present invention to disclose the aforementioned protocol wherein any of said selected Frequency is delivered with its corresponding overtones or multiples thereof.

It is an object of the present invention to disclose the aforementioned protocol wherein said AIVs amplitudes are selected on the basis of clinical efficacy.

It is an object of the present invention to disclose the aforementioned protocol wherein said volumes are selected on the basis of clinical efficacy.

It is an object of the present invention to disclose the aforementioned protocol wherein said sequences of different AIVs is accompanied by at least one set of predetermined humming therapeutic oscillations said humming therapeutic oscillations provided independently or contemporaneously with said series of AIVs.

It is an object of the present invention to disclose the aforementioned protocol wherein said sequences of different AIVs are accompanied by compression and/or massaging of at least one body portion of a patient in a predetermined manner.

It is an object of the present invention to disclose the aforementioned protocol wherein said vibrations are modulated according to a function selected from the group consisting of:

P (t)=ASin⁻¹ (Sin (ω₁t))+Bθ(Sin (ω₂t))+C,

P (t)=Floor (ASin⁻¹ (Sin (ω₁t)))+Bθ(Sin (ω₂t))+C,

P (t) A|Sin (ω₁t)|+C,

P (t)=ASin (ω₁t+σ₁)θ(Sin(ω₂t+σ₂))+C

It is an object of the present invention to disclose the aforementioned protocol wherein

0.01 mm≦A≦10 mm, 0.01 mm≦B≦10 mm, 0.01 mm≦C≦10 mm, 0.01 Hz≦ω₁≦100 Hz, 0.01 Hz≦ω₂≦100 Hz

It is an object of the present invention to provide an artificially induced vibrations device (FIG. 5) (AIVD) for increasing transcutaneous oxygen in a body portion of a patient said AIVD comprising:
a. a plurality of regulators (510), each of which is adapted to define an AIV with an individual frequency, pressure amplitude, volume and duration;
b. means for producing the protocol (520) of said defined individual AIVs; and,
c. effector (530) adapted to introduce said protocol of produced AIVs to impinge in or on at least at least one body portion of a patient;
   wherein said therapeutic protocol is effective in increasing transcutaneous oxygen in a body portion of a patient.

It is an object of the present invention to provide the aforementioned artificially induced vibrations device wherein said AIVD is additionally provided with means for producing at least one set of predetermined humming therapeutic oscillations said humming therapeutic oscillations provided independently or contemporaneously with said series of AIVs

It is an object of the present invention to provide the aforementioned artificially induced vibrations device wherein said AIVD is additionally provided with means for massaging and/or compressing at least one body portion of a patient in a predetermined manner.

It is an object of the present invention to provide the aforementioned artificially induced vibrations device wherein said AIVs are characterized by parameters selected from a group consisting of Frequency, Pressure, amplitude, Volume and Duration.

It is an object of the present invention to provide the aforementioned artificially induced vibrations device wherein said Frequency of AIVs is selected from a group consisting of about 0.5 Hz to about 5.0 Hz, about 5.0 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, any integer multiples thereof.

It is an object of the present invention to provide the aforementioned artificially induced vibrations device wherein said protocol is dependent upon said patient's natural involuntary or voluntary functions.

It is an object of the present invention to provide the aforementioned artificially induced vibrations device wherein any of said selected Frequency is deliverable with its corresponding overtones or multiples thereof

It is an object of the present invention to provide the aforementioned artificially induced vibrations device wherein said AIVs amplitudes are selectable on the basis of clinical efficacy

It is an object of the present invention to provide the aforementioned artificially induced vibrations device wherein said volumes are selectable on the basis of clinical efficacy

It is an object of the present invention to provide the aforementioned artificially induced vibrations device wherein said AIVD is adapted for external use on a patient's body

It is an object of the present invention to provide the aforementioned artificially induced vibrations device wherein said AIVD is adapted for internal use on a patient's body

It is an object of the present invention to provide the aforementioned artificially induced vibrations device wherein said AIVD is additionally provided with means for drug delivery.

It is an object of the present invention to provide the aforementioned artificially induced vibrations device wherein said therapeutic protocol comprises at least one individual AIV.

It is an object of the present invention to provide the AIVD wherein said AIVD is adapted for administering said protocol to said patients respiratory system

It is an object of the present invention to provide the aforementioned artificially induced vibrations device wherein said AIVD is adapted for administering said protocol to said patients circulatory system

It is an object of the present invention to provide the aforementioned artificially induced vibrations device wherein said AIVD is adapted for administering said protocol to said patients lymphatic system

It is an object of the present invention to provide the aforementioned artificially induced vibrations device wherein said AIVD is adapted for administering said protocol to said patient's cardiopulmonary system

It is an object of the present invention to disclose a protocol of artificially induced vibrations (AIVs) useful for increasing transcutaneous oxygen in a body portion of a patient said protocol comprising sequences of different AIVs; wherein said protocol is administered to at least one body portion of the patient; further wherein said sequences are independent of said patient's natural involuntary or voluntary functions.

It is an object of the present invention to provide an artificially induced vibrations device (FIG. 5) (AIVD) for clearing carbon dioxide from tissues of a treated body portion of a patient said AIVD comprising:
a. a plurality of regulators (510), each of which is adapted to define an AIV with an individual frequency, pressure amplitude, volume and duration;
b. means for producing the protocol (520) of said defined individual AIVs; and,
c. effector (530) adapted to introduce said protocol of produced AIVs to impinge in or on at least at least one body portion of a patient;
   wherein said therapeutic protocol is effective in increasing transcutaneous oxygen in a body portion of a patient .

It is an object of the present invention to provide the aforementioned AIVD a wherein said AIVD is additionally provided with means for producing at least one set of predetermined humming therapeutic oscillations said humming therapeutic oscillations provided independently or contemporaneously with said series of AIVs

It is an object of the present invention to provide the aforementioned AIVD wherein said AIVD is additionally provided with means for massaging and/or compressing at least one body portion of a patient in a predetermined manner.

It is an object of the present invention to provide the aforementioned AIVD wherein said AIVs are characterized by parameters selected from a group consisting of Frequency, Pressure, amplitude, Volume and Duration.

It is an object of the present invention to provide the aforementioned AIVD wherein said Frequency of AIVs is selected from a group consisting of about 0.5 Hz to about 5.0 Hz, about 5.0 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, any integer multiples thereof.

It is an object of the present invention to provide the aforementioned AIVD wherein said protocol is dependent upon said patient's natural involuntary or voluntary functions.

It is an object of the present invention to provide the aforementioned AIVD wherein any of said selected Frequency is deliverable with its corresponding overtones or multiples thereof

It is an object of the present invention to provide the aforementioned AIVD wherein said AIVs amplitudes are selectable on the basis of clinical efficacy

It is an object of the present invention to provide the aforementioned AIVD wherein said volumes are selectable on the basis of clinical efficacy

It is an object of the present invention to provide the aforementioned AIVD wherein said AIVD is adapted for external use on a patient's body

It is an object of the present invention to provide the aforementioned AIVD wherein said AIVD is adapted for internal use on a patient's body

It is an object of the present invention to provide The AIVD according to claim 64 wherein said AIVD is additionally provided with means for drug delivery.

It is an object of the present invention to provide the aforementioned AIVD wherein said therapeutic protocol comprises at least one individual AIV.

It is an object of the present invention to provide the aforementioned AIVD wherein said AIVD is adapted for administering said protocol to said patients respiratory system

It is an object of the present invention to provide the aforementioned AIVD wherein said AIVD is adapted for administering said protocol to said patients circulatory system

It is an object of the present invention to provide the aforementioned AIVD wherein said AIVD is adapted for administering said protocol to said patients lymphatic system

It is an object of the present invention to provide the aforementioned AIVD wherein said AIVD is adapted for administering said protocol to said patient's cardiopulmonary system

It is an objective of the present invention to disclose a protocol of artificially induced vibrations (AIVs) useful for for clearing carbon dioxide from tissues of a treated body portion of a patient said protocol comprising sequences of different AIVs; wherein said protocol is administered to at least one body portion of the patient; further wherein said sequences are independent of said patient's natural involuntary or voluntary functions.

It is an objective of the present invention to disclose a protocol wherein said protocol is dependent upon said patient's natural involuntary or voluntary functions.

It is an objective of the present invention to disclose a protocol wherein said AIVs are transmitted to said at least one body portion of a patient by pneumatic means

It is an objective of the present invention to disclose a protocol wherein said AIVs are transmitted to said at least one body portion of a patient by mechanical means

It is an objective of the present invention to disclose a protocol wherein said AIVs are transmitted to said at least one body portion of a patient by hydraulic means

It is an objective of the present invention to disclose a protocol wherein said AIVs are transmitted to said at least one body portion of a patient by electrical means

It is an objective of the present invention to disclose a protocol T wherein said AIVs are transmitted to said at least one body portion of a patient by ultrasound means

It is an objective of the present invention to disclose a protocol wherein said AIVs are characterized by parameters selected from a group consisting of Frequency, Pressure, amplitude, Volume and Duration.

It is an objective of the present invention to disclose a protocol wherein said Frequency of AIVs is selected from a group consisting of about 0.5 Hz to about 5.0 Hz, about 5.0 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, any integer multiples thereof.

It is an objective of the present invention to disclose a protocol wherein any of said selected Frequency is delivered with its corresponding overtones or multiples thereof

It is an objective of the present invention to disclose a protocol wherein said AIVs amplitudes are selected on the basis of clinical efficacy

It is an objective of the present invention to disclose a protocol wherein said volumes are selected on the basis of clinical efficacy

It is an objective of the present invention to disclose a protocol wherein said sequences of different AIVs is accompanied by at least one set of predetermined humming therapeutic oscillations said humming therapeutic oscillations provided independently or contemporaneously with said series of AIVs

It is an objective of the present invention to disclose a protocol wherein said sequences of different AIVs are accompanied by compression and/or massaging of at least one body portion of a patient in a predetermined manner

It is an objective of the present invention to provide an artificially induced vibrations device (FIG. 5) (AIVD) for controlling NO production in tissues of a treated body portion of a patient said AIVD comprising:
a. a plurality of regulators (510), each of which is adapted to define an AIV with an individual frequency, pressure amplitude, volume and duration;
b. means for producing the protocol (520) of said defined individual AIVs; and,
c. effector (530) adapted to introduce said protocol of produced AIVs to impinge in or on at least at least one body portion of a patient;
   wherein said therapeutic protocol is effective in increasing transcutaneous oxygen in a body portion of a patient.

It is an objective of the present invention to provide the aforementioned AIVD according to claim wherein said AIVD is additionally provided with means for producing at least one set of predetermined humming therapeutic oscillations said humming therapeutic oscillations provided independently or contemporaneously with said series of AIVs

It is an objective of the present invention to provide the aforementioned AIVD wherein said AIVD is additionally provided with means for massaging and/or compressing at least one body portion of a patient in a predetermined manner.

It is an objective of the present invention to provide the aforementioned AND wherein said AIVs are characterized by parameters selected from a group consisting of Frequency, Pressure, amplitude, Volume and Duration.

It is an objective of the present invention to provide the aforementioned AIVD wherein said Frequency of AIVs is selected from a group consisting of about 0.5 Hz to about 5.0 Hz, about 5.0 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, any integer multiples thereof.

It is an objective of the present invention to provide the aforementioned AIVD wherein said protocol is dependent upon said patient's natural involuntary or voluntary functions.

It is an objective of the present invention to provide the aforementioned AIVD wherein any of said selected Frequency is deliverable with its corresponding overtones or multiples thereof

It is an objective of the present invention to provide the aforementioned AIVD wherein said AIVs amplitudes are selectable on the basis of clinical efficacy

It is an objective of the present invention to provide the aforementioned AIVD wherein said volumes are selectable on the basis of clinical efficacy

It is an objective of the present invention to provide the aforementioned AIVD wherein said AIVD is adapted for external use on a patient's body

It is an objective of the present invention to provide the aforementioned AND wherein said AIVD is adapted for internal use on a patient's body

It is an objective of the present invention to provide the aforementioned AIVD wherein said AIVD is additionally provided with means for drug delivery.

It is an objective of the present invention to provide the aforementioned AIVD wherein said therapeutic protocol comprises at least one individual AIV.

It is an objective of the present invention to provide the aforementioned AIVD wherein said AIVD is adapted for administering said protocol to said patients respiratory system

It is an objective of the present invention to provide the aforementioned AIVD wherein said AIVD is adapted for administering said protocol to said patients circulatory system

It is an objective of the present invention to provide the aforementioned AIVD wherein said AIVD is adapted for administering said protocol to said patients lymphatic system

It is an objective of the present invention to provide the aforementioned AIVD wherein said AIVD is adapted for administering said protocol to said patient's cardiopulmonary system

It is an objective of the present invention to disclose a protocol of artificially induced vibrations (AIVs) useful for controlling NO production in tissues of a treated body portion of a patient said protocol comprising sequences of different AIVs; wherein said protocol is administered to at least one body portion of the patient; further wherein said sequences are independent of said patient's natural involuntary or voluntary functions.

It is an objective of the present invention to disclose the aforementioned protocol wherein said protocol is dependent upon said patient's natural involuntary or voluntary functions.

It is an objective of the present invention to disclose the protocol wherein said AIVs are transmitted to said at least one body portion of a patient by pneumatic means

It is an objective of the present invention to disclose the aforementioned protocol wherein said AIVs are transmitted to said at least one body portion of a patient by mechanical means

It is an objective of the present invention to disclose the aforementioned protocol wherein said AIVs are transmitted to said at least one body portion of a patient by hydraulic means

It is an objective of the present invention to disclose the aforementioned protocol wherein said AIVs are transmitted to said at least one body portion of a patient by electrical means

It is an objective of the present invention to disclose the aforementioned protocol wherein said AIVs are transmitted to said at least one body portion of a patient by ultrasound means

It is an objective of the present invention to disclose the aforementioned protocol wherein said AIVs are characterised by parameters selected from a group consisting of Frequency, Pressure, amplitude, Volume and Duration.

It is an objective of the present invention to disclose the aforementioned protocol wherein said Frequency of AIVs is selected from a group consisting of about 0.5 Hz to about 5.0 Hz, about 5.0 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, any integer multiples thereof.

It is an objective of the present invention to disclose the aforementioned protocols wherein any of said selected Frequency is delivered with its corresponding overtones or multiples thereof

It is an objective of the present invention to disclose the aforementioned protocols wherein said AIVs amplitudes are selected on the basis of clinical efficacy

It is an objective of the present invention to disclose the aforementioned protocols wherein said volumes are selected on the basis of clinical efficacy

It is an objective of the present invention to disclose the aforementioned protocol wherein said sequences of different AIVs is accompanied by at least one set of predetermined humming therapeutic oscillations said humming therapeutic oscillations provided independently or contemporaneously with said series of AIVs

It is an objective of the present invention to disclose the aforementioned protocol wherein said sequences of different AIVs are accompanied by compression and/or massaging of at least one body portion of a patient in a predetermined manner

It is also objective of the present invention to disclose a method useful for increasing angiogenesis and/or capillary sprout growth in a body portion of a mammalian patient, said method comprising steps of
i. obtaining an artificially induced vibrations device (FIG. 1) (AIVD) for increasing angiogenesis and/or capillary sprout growth in a body portion of a patient said AIVD comprising:
a. a plurality of regulators (510), each of which is adapted to define an AIV with an individual frequency, pressure amplitude, volume and duration;
b. means for producing the protocol (520) of said defined individual AIVs; and,
c. effector (530) adapted to introduce said protocol of produced AIVs to impinge in or on at least at least one body portion of a patient;
   wherein said therapeutic protocol is effective in increasing transcutaneous oxygen in a body portion of a patient and
ii. operating said AIVD on said patient .

It is an objective of the present invention to disclose protocol of artificially induced vibrations (AIVs) useful for increasing angiogenesis and/or capillary sprout growth in tissues of a treated body portion of a patient said protocol comprising sequences of different AIVs; wherein said protocol is administered to at least one body portion of the patient; further wherein said sequences are independent of said patient's natural involuntary or voluntary functions.

It is an objective of the present invention to disclose a method useful for increasing angiogenesis and/or capillary sprout growth in a body portion of a mammalian patient, said method comprising steps of
i. obtaining an artificially induced vibrations device (FIG. 5) (AIVD) for increasing perfusion in a body portion of a patient said AIVD comprising:
a. a plurality of regulators (510), each of which is adapted to define an AIV with an individual frequency, pressure amplitude, volume and duration;
b. means for producing the protocol (520) of said defined individual AIVs; and,
c. effector (530) adapted to introduce said protocol of produced AIVs to impinge in or on at least at least one body portion of a patient;
   wherein said therapeutic protocol is effective in increasing transcutaneous oxygen in a body portion of a patient and
ii operating said AIVD on a body portion of said patient.

It is an objective of the present invention to disclose a protocol of artificially induced vibrations (AIVs) useful for increasing perfusion in tissues of a treated body portion of a patient said protocol comprising sequences of different AIVs; wherein said protocol is administered to at least one body portion of the patient; further wherein said sequences are independent of said patient's natural involuntary or voluntary functions.

It is an objective of the present invention to disclose a method for increasing perfusion in at least a portion of a heart of a mammalian patient said AIVD comprising obtaining a non-invasive rigid-support enhanced external counterpulsation device (RS-EECP) providing a precise onset of a blood flow characterized by a sharp-wave front, useful for out-patient treatment of arterial insufficiency states, especially angina; said RS-EECP comprising a timing means and a plurality of pressing cuffs; said timing means is adapted to onset the collapsing and expanding maneuvers of the cuffs in a sequence of occasions defined along the diastolic/systolic cycle; said cuffs are fastened around at least a portion of the circumference of at least one organ comprising a vascular bed to counterpulsate against an either fixed or maneuverable support; wherein said support is a rigid member; such that a quick expansion of said vessel bed, following a forceful and effective collapsing of the same is obtained further wherein said (RS-EECP) is adapted to provide a protocol of artificially induced vibrations (AIVs) useful for increasing perfusion in tissues of a treated body portion of a patient said protocol comprising sequences of different AIVs; wherein said protocol is administered to at least one body portion of the patient; further wherein said sequences are independent of said patient's natural involuntary or voluntary functions and
ii. operating said RS-EECP on said mammalian patient.

The aforementioned protocols of the present invention can be in a certain ratio in time with diastole and/or systole.

It is a further objective of the present invention to disclose a non-invasive method for out-patient treating of arterial insufficiency states, especially angina by providing a precise onset of a blood flow characterized by a sharp pressure wavefront with the use of the aforementioned RS-EECP.

It is an objective of the present invention to disclose the aforementioned method, comprising steps of:
a. obtaining a timing means and a plurality of pressing cuffs;
b. fastening cuffs around at least a portion of the circumference of at least one organ comprising a vascular bed to counterpulsate against an either fixed or maneuverable support; wherein said support is a rigid member;
c. initiating the collapsing and expanding maneuvers of the cuffs in a sequence of occasions defined along the diastolic/systolic cycle;
such as a quick expansion of said vessel bed, following a forceful and effective collapsing of the same is obtained.

It is an objective of the present invention to disclose the aforementioned method useful for out-patient treating of arterial insufficiency states, comprising at least one step of providing a precise onset of a blood flow characterized by a sharp-wave front, comprising providing a portable CPR device.

It is an objective of the present invention to disclose the aforementioned method further comprising obtaining a synergic resuscitating system comprising CPR and a defibrillator; utilizing said portable CPR in an independent manner, in a conjunction and/or in a communication with said defibrillator.

It is an objective of the present invention to disclose the aforementioned method comprising monitoring said synergic resuscitating system with at least one controlling means.

It is an objective of the present invention to disclose the aforementioned method where said pressure is varied according to an equation selected from the group consisting of:

P(t) Asin⁻¹ (sin(ω₁t))+B×θ(sin(ω₂t))+C

where θ(t) is the Heaviside step function

$$θ ( x ) = \{ \begin{matrix}1 ; & x \geq 0 \\ 0 ; & x < 0 ;\end{matrix} \\ e . P ( t ) = A {sin}^{- 1} ( sin ( {ω}_{1} t ) ) + B \times θ ( sin ( {ω}_{2} t ) ) + C \\ f . P ( t ) = A {sin}^{- 1} ( sin ( {ω}_{1} T ) ) + B {sin}^{- 1} ( sin ( {ω}_{2} t ) ) + C \\ g . P ( t ) = {P}_{0} erfc ( \frac{{l}^{2}}{Dt} )$$

It is an objective of the present invention to disclose the aforementioned method where the constants A, B, C, ω₁, and ω₂ are such that 0.01 Hz≦ω₁≦1 Hz, 0.1 Hz≦ω₂≦10 Hz, 10 mmHg≦A≦250 mmHg, 10 mmHg≦B≦250 mmHg, and −250 mmHg≦C≦250 mmHg (see FIGS. 4a, 4b, 4c, 4d, 4e).

It is an objective of the present invention to disclose a method of treating nasal congestion and/or relieving sinusitis symptoms in a mammalian patient, comprising attaching a vibration generating means artificially induced vibrations device (FIG. 1) (AIVD) to the patient head, at a location adjacent to sinuses to be treated, generating a vibration by said vibration generating means, and delivering the same to said patient wherein said artificially induced vibrations device (FIG. 5) (AIVD) produces artificially induced vibrations of any of a given frequency, pressure, amplitude, volume and duration according to a protocol; said AIVD comprising:
a. a plurality of regulators (510), each of which is adapted to define an AIV with an individual frequency, pressure amplitude, volume and duration;
b. means for producing the protocol (520) of said defined individual AIVs; and,
c. effector (530) adapted to introduce said protocol of produced AIVs to impinge in or on at least at least one body portion of a patient;
   wherein said therapeutic protocol of AIVs comprises two or more different individual time resolved AIVs.

It is an objective of the present invention to disclose a method wherein the location adjacent to sinuses to be treated are adjacent to nasal cavity, nasal passageway, sinus or sinuses of the patient and are selected from the nasal bone, root-, dorsum- or bridge- of the nose, nostrils, frontal bone, temporal bone, maxilla bone, superciliary arch or any combination thereof.

It is an objective of the present invention to disclose the aforementioned method wherein delivering the vibration is applied by cycles of intermittent vibrations or pulses and/or by applying vibration of various amplitudes.

It is an objective of the present invention to disclose the aforementioned method additionally comprising pulsating a stream of fluid towards the patient's respiration tracts wherein the fluid is air or a mixture of air and at least one liquid or gas or powder.

It is an objective of the present invention to disclose the aforementioned method additionally comprising vaporizing essential oils, volatile compounds, etheric oils, terepenes, terpanols and either water miscible or water-immiscible extracts, especially oils or extracts obtained from Amyris, Balsam, Bay Rum, Black Pepper, bomyl acetate, Cajeput, Camphor, Cedarwood, Cedarleaf oil, Chamomile, chlorbutanol, Cinnamon, Clary Sage Rosewood, Clove, Eucalyptus, Frankincense, Geranium, Ginger, Lavender, Lemon, Lemon essential oils, levomenthol, Lime, Menthol, Mint, Myrrh, nutmeg oil Orange, Patchouli, Peppermint, Pine Needle, Rose Eucalyptus, rosemary, Rosewood, Sage, Sandalwood, Spearmint, Tea Tree, terpinol, turpentine oil, thymol, Ylang Ylang or any combination thereof.

It is an objective of the present invention to disclose the aforementioned method additionally comprising vaporizing at least one of the group of Braochodilators, especially sympatic mimetics, alfa antagonists, anti cholinergics; nasal decongestants, such as pseudoehedrines, ephedrines; steroids; anti histamines; anti prostaglandins, alternative or homeopathic medicaments; vaso constrictors; local anesthetics; mast cell stabilizers; antibiotics, such as biocides, fungicides etc; pleasant odor; pheromones; hormone treatments, such as ADH, insulin, growth hormones; vapors, humidifiers; drying compositions; hot or cold vapors; hyper-, iso- or hypotonic vapors or any combination thereof.

It is an objective of the present invention to disclose the aforementioned method additionally comprising vaporizing at least one of the group of compositions and commercial available medicaments, their derivatives, or by-products provided thereof, selected from Acrivastine, Aller-Eze Clemastine, Aller-Eze nasal spray, Azatadine maleate, Azelastine nasal spray, Beclometasone nasal spray, Beclometasone nasal spray, Beconase hayfever nasal spray, Beconase hayfever relief for adults, Beconase nasal spray, Benadryl allergy relief, Benadryl, Benadryl, Benadryl plus, Brompheniramine maleate, Budesonide nasal spray, Calimal Antihistamine, Cetirizine, Chlorphenamine, Clarityn, Clemastine, Cyproheptadine hydrochloride, Desloratadine, Dexa-Rhinaspray Duo. Dimotane elixir, Dimotane plus, Dimotapp elixir, Dimotapp elixir paediatric. Dimotapp LA, Flixonase allergy nasal spray, Flixonase aqueous nasal spray, Fluticasone propionate nasal spray, Galpharm hayfever and allergy relief, Galpseud Plus, Haymine , Histafen, Ipratropium bromide nasal spray, Levocabastine nasal spray, Levocetirizine dihydrochloride, Livostin direct nasal spray, Livostin nasal spray, Loratadine, Medised, Medised, Mistamine, Mizolastine, Mizollen, Mometasone furoate nasal spray, Nasacort, Nasobec nasal spray, Nasonex nasal spray, Neoclarityn tablets/syrup, Optimine syrup, Periactin, Phenergan, Piriteze, Piriton, Pollenase hayfever nasal spray, Promethazine hydrochloride elixir, Promethazine hydrochloride, Rhinocort Aqua, Rhinolast allergy nasal spray, Rhinolast nasal spray, Rinatec nasal spray, Rino clenil nasal spray, Rynacrom allergy nasal spray, Rynacrom nasal spray, Semprex, Sodium cromoglicate nasal spray, Sudafed Plus, Syntaris nasal spray, Tavegil, Telfast 120, Terfenadine, Terfinax, Triamcinolone acetonide, Vista-Methasone, Xyzal tablets, Zirtek allergy relief tablets, Zirtek allergy tablets/solution, Afrazine, Anadin, Beechams all-in-one, Beechams products, Benylin products, Contac, Day Nurse, Dimotapp elixir, Dimotapp elixir paediatric, Dimotapp products, Galpseud, Karvol decongestant products, Lemsip products, Meggezones, Merocets Plus lozenges, Nurofen Cold and Flu, Otrivine Menthol Nasal Spray, Otrivine Metered Dose Sinusitis Spray, Otrivine products, Pseudoephedrine hydrochloride, Sterwin real lemon cold powders and other products, Strepsils Menthol and Eucalyptus and other products, Sudafed and its products, Xylometazoline nasal drops, Bactroban Nasal, Fusafungine, Locabiotal, Naseptin nasal cream, Ipratropium bromide nasal spray, Rinatec nasal spray, pseudoephedrine, propylhexedrine, L-Desoxyephedrine, xylometazoline hydrochloride or any combination thereof.

It is an objective of the present invention to disclose the aforementioned method additionally comprising cooling or heating said fluid before or in the process of forcing it towards the respiratory tracks.

It is an objective of the present invention to disclose the aforementioned method additionally comprising activating a medicament by reacting it with one or more reactants before or in the process of forcing the same towards the respiratory tracks.

It is an objective of the present invention to disclose the aforementioned method additionally comprising inhaling a dispersed medicament.

It is an objective of the present invention to disclose a method for treating treating nasal congestion and/or relieving sinusitis symptoms in a mammalian patient, comprising attaching means and a vibration generating means; said attaching means are in communication with the patient's head in a location adjacent to said nasal cavity, nasal passageway or sinuses to be treated; said generating means are adapted to vibrate said attaching means, to vibrate the location adjacent to said nasal cavity or sinuses and thus improved mucociliary clearance of secretions in nasal cavity or sinuses such a device includes a mechanism or mechanisms adapted to provide said therapeutic protocol of AIVs comprises two or more different individual time resolved AIVs

It is an objective of the present invention to disclose the aforementioned method wherein the vibrating means comprises a motor, a power source, at least one wheel for transporting the power generated by the motor to a circular movement rotating cogwheels and at least one weight, the position there between generates a torque translated into vibrations to be transported to a plate or membrane.

It is an objective of the present invention to disclose the aforementioned method, wherein said cogwheels hit a rigid material with at least every one rotation, the impact on the rigid material generates vibrations to be transported to a plate or membrane.

It is an objective of the present invention to disclose the aforementioned method wherein the vibrating means comprises a motor, a power source and a rotor in communication with an axle rotating coaxially, said rotation being translated to a vibration and further being delivered to body portion adjacent sinuses of the patient.

It is an objective of the present invention to disclose the aforementioned method wherein the vibrating means comprising an electrical or electro-magnetic motor.

It is an objective of the present invention to disclose the aforementioned method additionally comprising means of forcing a stream of fluid towards the patient's respiration tracks and/or medicament dispersing means further wherein the medicament delivery is provided by 2D or 3D specific and predetermined fitting mechanism between the capsule and the device's envelope, or by any other physical parameter.

It is an objective of the present invention to disclose the aforementioned method wherein the fluid is air or a mixture of air and at least one liquid, gas or solid powder other than air.

It is an objective of the present invention to disclose the aforementioned method additionally comprising heating means adapted to heat said fluid before or in the process of forcing it towards the respiratory tracts.

It is an objective of the present invention to disclose the aforementioned method additionally comprising activating means adapted to activate a medicament by reacting it with one or more reactants before or in the process of forcing the same towards the respiratory tracts.

It is an objective of the present invention to disclose the aforementioned method wherein the medicament is at least partially contained in a capsule.

It is an objective of the present invention to disclose the aforementioned method additionally comprising one or more dispensing means being either active or passive means, adapted to force or purge at least a portion of the dispersed material towards a predetermined, e.g., focused target being either adjacent to the invented device or located in a remote location.

It is an objective of the present invention to disclose the aforementioned method wherein the amplitude of said vibrations are modulated according to an equation selected from the group consisting of:

h. P(t)=Asin⁻¹ (sin (ω₁t)+B×θ(sin (ω₂t))+C

where θ(t) is the Heaviside step function

$$θ ( x ) = \{ \begin{matrix}1 ; & x \geq 0 \\ 0 ; & x < 0 ;\end{matrix} \\ i . P ( t ) = A {sin}^{- 1} ( sin ( {ω}_{1} t ) ) + B \times θ ( sin ( {ω}_{2} t ) ) + C \\ j . P ( t ) = A {sin}^{- 1} ( sin ( {ω}_{1} T ) ) + B {sin}^{- 1} ( sin ( {ω}_{2} t ) ) + C \\ k . P ( t ) = {P}_{0} erfc ( \frac{{l}^{2}}{Dt} )$$

It is an objective of the present invention to disclose the aforementioned method wherein the constants A, B, C, ω₁, and ω₂ are such that 0.01 Hz≦ω₁≦1 Hz , 0.1 Hz≦ω₂≦10 Hz , 10 mmHg≦A≦250 mmHg , 10 mmHg≦B≦250 mmHg, and −250 mmHg≦C≦250 mmHg.

It is an objective of the present invention to disclose the aforementioned method wherein said artificially induced vibrations (AIVs) are administered as a protocol comprising sequences of different AIVs; wherein said protocol is administered to at least one body portion of the patient; further wherein said AIVs are in the form of pulsating negative pressures, or pulsating negative and positive pressures.

It is an objective of the present invention to disclose the aforementioned method wherein said pulsating negative pressures, or pulsating negative and positive pressures are produced in the respiratory system.

It is an objective of the present invention to disclose the aforementioned method of wherein said pulsating negative pressures, or pulsating negative and positive pressures are produced in the respiratory system of a mammalian patient.

It is an objective of the present invention to disclose the aforementioned method of wherein said pulsating negative pressures, or pulsating negative and positive pressures are produced in the circulatory system of a mammalian patient.

It is an objective of the present invention to disclose the aforementioned method of wherein said pulsating negative pressures, or pulsating negative and positive pressures are produced in the cardiovascular system of a mammalian patient.

### BRIEF DESCRIPTION OF THE DRAWINGS

FIG. 1 is a schematic illustration of an aspect of the present invention

FIG. 2 is a schematic illustration of an aspect of the present invention

FIG. 3 are schematic illustration of an aspect of the present invention

FIG. 4 are schematic illustration of an aspect of the present invention

FIG. 5 is a schematic illustration of an aspect of the present invention

### DETAILED DESCRIPTION OF THE FIGURES

The following description is provided, alongside all chapters of the present invention, so as to enable any person skilled in the art to make use of said invention and sets forth the best modes contemplated by the inventor of carrying out this invention. Various modifications, however, are adapted to remain apparent to those skilled in the art, since the generic principles of the present invention have been defined specifically to provide an air delivery device and method of using the same.

It is a core principle of the present invention that vibrational impacts, which make contact with mammalian cells, tissue, organs or systems induces a response from the target. It is further known that the responses vary according to the strength of the impact vibration, acceleration, intensity etc. The impacts may be direct mechanical impacts, or hydraulically mediated through fluid, or through direct mechanical contact, or via electromagnetic waves. The present invention provides means, methods and protocols for improving a patient's wellness by the application of predetermined Artificially induced Vibrations (AIVs) to mammalian patients. It should be noted that all embodiments of the present invention provide the Artificially induced Vibrations (AIVs) administered as contacting impacts with a bodily portion of the subject. In certain embodiments, especially but not exclusively directed towards any part of the respiratory system, or for wound healing, these impacts may be indirect in the sense that they are produced by air pressure waves which have been generated by an air pressure wave generator. and the scope of the invention emphatically excludes sound waves or music or aural stimulations or sonic waves contact, or via electromagnetic waves. The present invention provides means, methods and protocols for improving a patient's wellness by the application of predetermined Artificially induced Vibrations (AIVs) to mammalian patients. It should be noted that all embodiments of the present invention provide the Artificially induced Vibrations (AIVs) administered as contacting impacts with a bodily portion of the subject. In certain embodiments, especially but not exclusively directed towards any part of the respiratory system, or for wound healing, these impacts may be indirect in the sense that they are produced by air pressure waves which have been generated by an air pressure wave generator. and the scope of the invention emphatically excludes sound waves or music or aural stimulations or sonic waves generated at a remote location and directed towards the mammalian patients ear or hearing system.

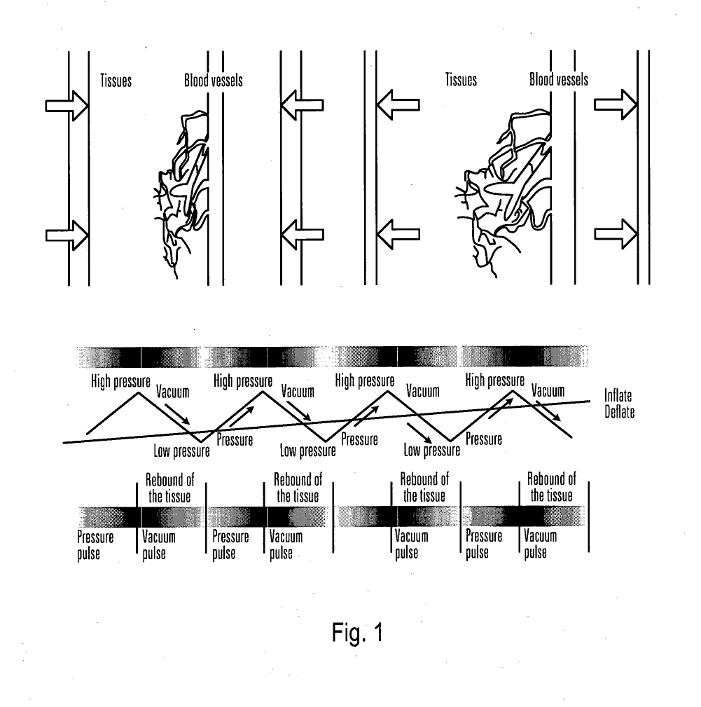

A major principle of the present invention are means and methods of inducing vacuum pulses, pressure pulses and alternating vacuum/pressure pulses on tissues, in order to exert physiological effects. It is herein acknowledged that in FIGS. 1a and 1b low pressure and negative pressure are synonymous.

The following documents are incorporated herein in a non limiting manner.

Application 60/991,772 titled SYSTEM FOR PREVENTING AND TREATING PAIN IN THE LUMBAR REGION AND ELSEWHERE IN THE HUMAN BODY

WO/2010/038233 titled PROTOCOL AND METHODS FOR PULSATING DRUG DELIVERY 61/250,526 titled FUNCTIONAL REVASCULARIZATION and 61/367,423 titled MEANS AND METHODS OF ALLEVIATING PERIPHERAL CIRCULATORY DISORDERS

WO/2006/129305 titled PULSATING INHALER AND A METHOD OF TREATING UPPER RESPIRATORY DISORDERS

WO/2007/046088 titled AN EECP DEVICE AND AN IMAGE SYSTEM COMPRISING THE SAME

In each of the above documents devices, means, mechanisms and embodiments are described in a non limiting manner which can have incorporated within them CPUs, controllers, programmable codes or computer implementation media or components which will enable them to operate the novel methods and protocols disclosed herein wherein said artificially induced vibrations (AIVs) are administered as a protocol comprising sequences of different AIVs; said protocol is administered to at least one body portion of the patient; wherein said AIVs are in the form of pulsating negative (low) pressures, or pulsating negative (low) and positive pressures wherein said protocol of AIVs comprises one, two or more different individual time resolved AIVs. FIG. 1 is illustrative; 110 shows the outcome in terms of capillary growth; 120 shows the pressure sequence and 130 shows the effect of tissue rebound, as, in this case, pressure and vacuum pulses are applied.

Some of the aforementioned methods and protocols are incorporated in the aforementioned embodiments such that the pulsating negative (low) pressures, or pulsating negative (low) and positive pressures are directed towards or produced in the respiratory system of a mammalian patient.

Some of the aforementioned methods and protocols are incorporated in the aforementioned embodiments such that the pulsating negative (low) pressures, or pulsating negative (low) and positive pressures are directed towards or produced in the lymphatic system of a mammalian patient.

Some of the aforementioned methods and protocols are incorporated in the aforementioned embodiments such that the pulsating negative (low) pressures, or pulsating negative (low) and positive pressures are directed towards or produced in the circulatory system of a mammalian patient.

Some of the aforementioned methods and protocols are incorporated in the aforementioned embodiments such that the pulsating negative (low) pressures, or pulsating negative (low) and positive pressures are directed towards or produced in the cardiovascular system of a mammalian patient.

The term ‘tcpO2’ hereinafter refers to transcutaneous oxygen partial pressure. The term ‘tcpCO2’ hereinafter refers to transcutaneous carbon dioxide partial pressure. (Transcutaneous Blood Gas Monitoring Hutch, A., Acta Anaesthesiologica Scandinavica. Supplementum. 107:87-90,1995) Transcutaneous blood gas measurement is a noninvasive, diagnostic technique that records the partial pressure of oxygen(PtcO2) and carbon dioxide (PtcCO2) electrochemically at the skin surface. It is noninvasive and is appropriate for continuous and prolonged monitoring. Three interdependent factors determine PtcO2 and PtcCO2 : Skin Perfusion, Skin Respiration and Skin Diffusion.

It has been postulated that angiogenesis and increased perfusion is promoted by deliberate deformation of tissues caused by application of vacuum, vibration and squeezing or massaging. In the present invention, vacuum, compression, vibration and squeezing or massaging or any other type of pressure perturbation may be carried out to such an extent that the difference between maximum and minimum may cover the range from above the normal basal level to the basal level, or above the normal basal level to below the basal level, thereby providing a different physiological effect. The stretching of cells encourages capillary sprouting and cellular proliferation. (see FIG. 1. 110, 120 130) Furthermore, evidence suggests that gene regulation and expression in vascular endothelial cells responds to mechanical forces exerted on the cells.

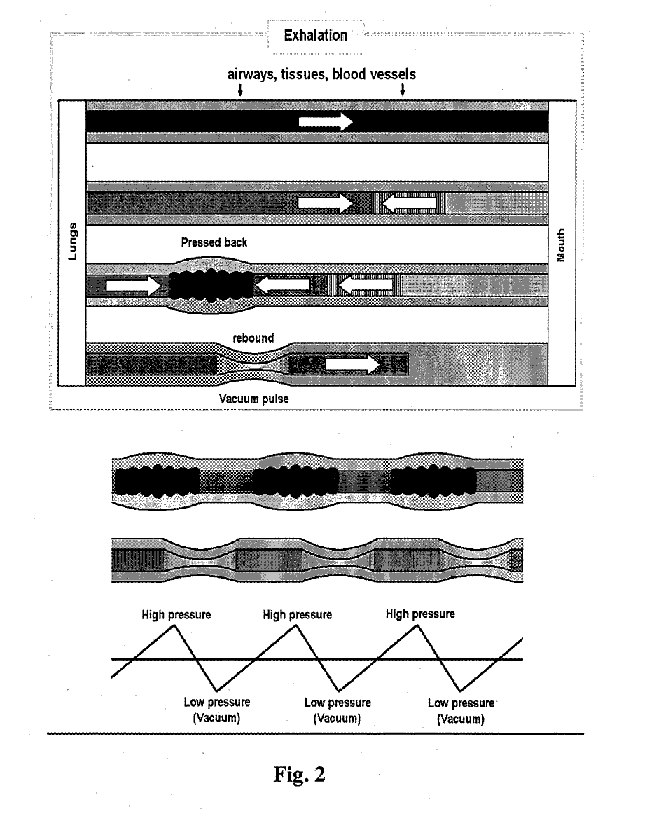

When a sudden or abrupt high pressure pulse impinges a tissue, the tissue is displaced (a), and a physiological response, change or effect is brought about. This response, change or effect is termed HPR. When the high pressure pulse is released, the tissue will relax and is no longer displaced. Another physiological response, change or effect at the cellular level or at the tissue level occurs as a consequence. Yet another HPR is envisaged if, instead of the exerted pressure dropping merely to the basal level, a vacuum pulse is exerted so that the tissue undergoes a larger pressure gradient from a positive pressure to a negative pressure, and a corresponding larger displacement and rebound (or spring-back) of the tissue, with enhanced HPR (FIG. 2). FIG. 210 schematically illustrates the rebound effect in airways, tissues and blood vessels. It is further acknowledged that methods, protocols and devices for inducing physiological response, change or effect by administration of pulsating negative pressures, or pulsating negative and positive pressures are novel apects of the present invention. It is the pulsating aspect of these different pressures which can, in some embodiments of the present invention, constitute novel and suprising protocols or sub protocols. wherein protocols or sub protocols of AIVs comprises one, two or more different individual time resolved AIVs (sequences of AIVs). It is herein acknowledged that humming sequences refer to drone like sequences of AIVs that can be part of a protocol. The concept of tissue rebound can be understood as the tendency, of a tissue to correct a displacement brought about by an applied pressure. In some protocols of the present invention, the natural frequencies and resonances of individual organs, systems or tissues are exploited.

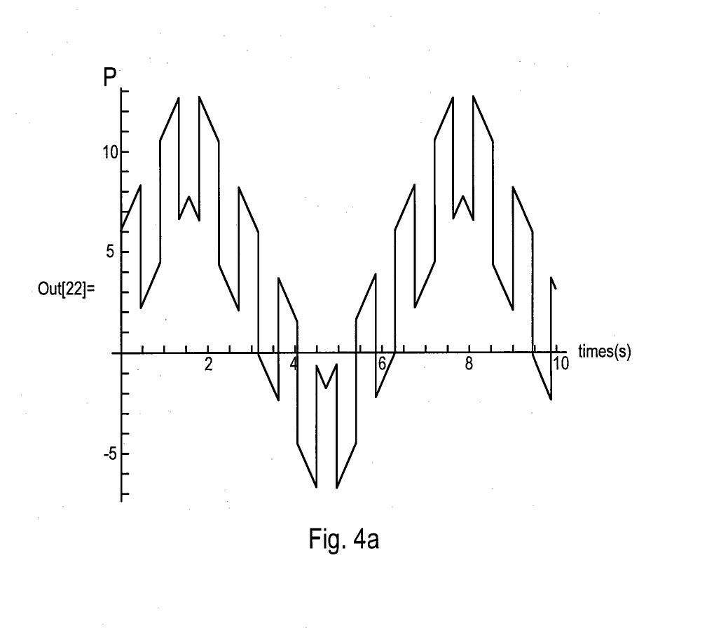

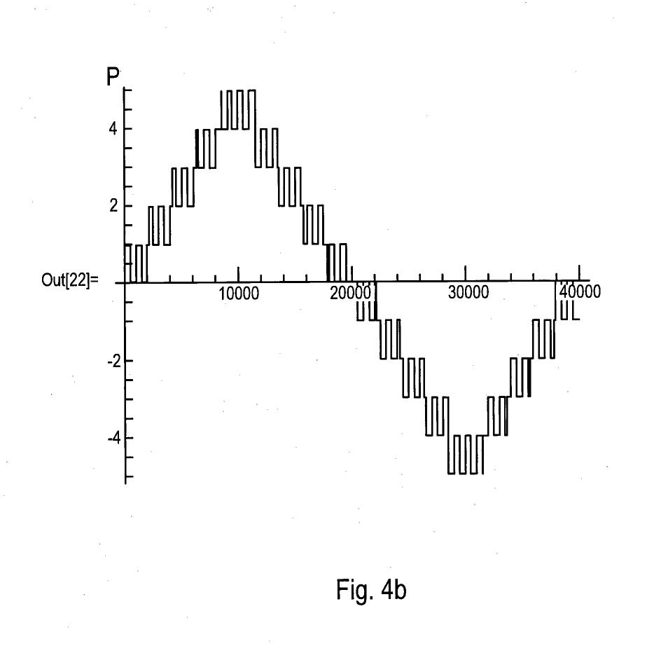

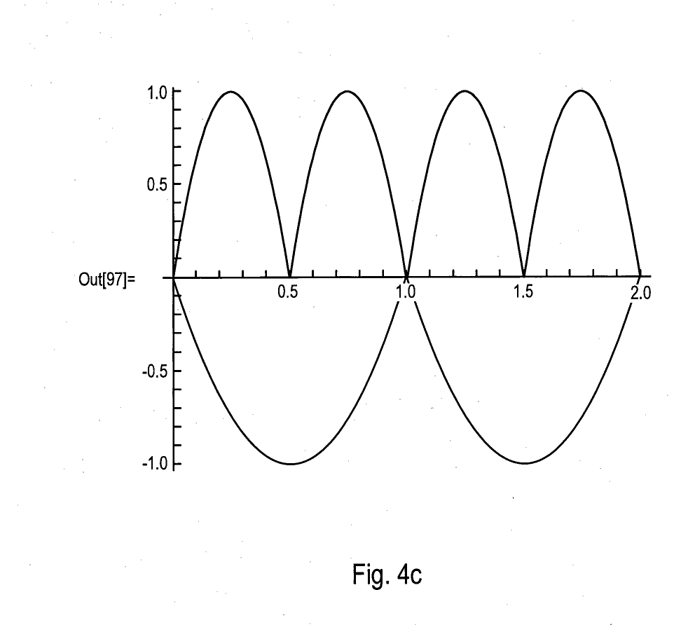

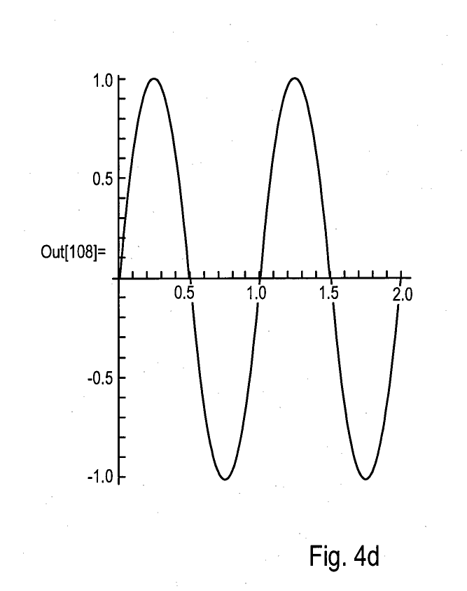

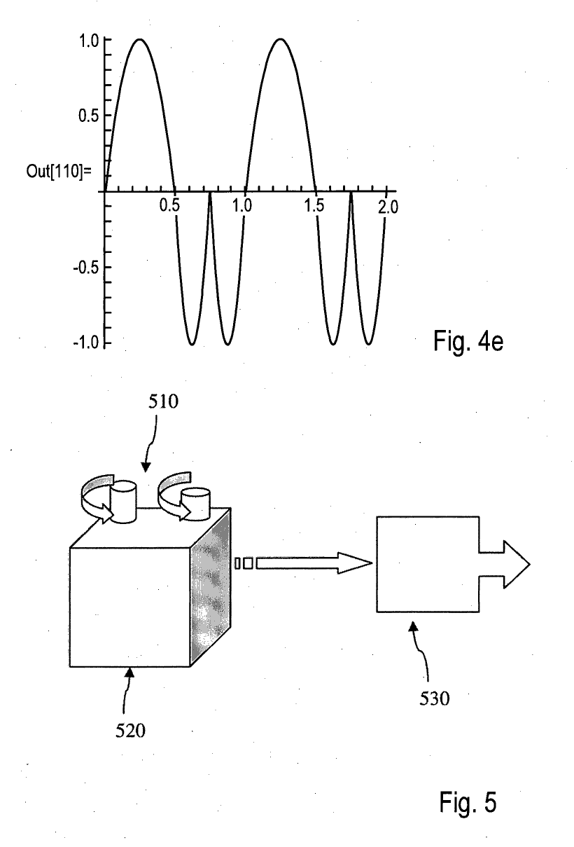

In some embodiments of the present invention, the aforementioned protocols, methods or devices for administration of pulsating negative pressures, or pulsating negative and positive pressures, when coupled with drug delivery to a tissue or organ or system are highly significant advances. Moreover, in the case of the respiratory system, it is a core of the pesent invention to provide the aforementioned protocols or subprotocols in combination with any type of inhaler, assisted breathing apparatus, CPAP device, vibrating device, drug delivery device or any other air or fluid delivery device. Fiurthermore, It is a core principle of the present invention to provide the aforementioned protocols or subprotocols in combination with any device acting in whole or in part upon the peripheral vascular system, cardiopulmonary system, venous system, arteries or lymphatic system. It is well within the scope of the present invention to provide the aforementioned methods and protocols for administration of pulsating negative pressures, or pulsating negative and positive pressures, delivered by a non-invasive rigid-support enhanced external counterpulsation device such as that provided in patent PCT/IL2006/001189 titled AN EECP DEVICE AND AN IMAGE SYSTEM COMPRISING THE SAME or any other device. FIGS. 2 schematically represent the changes in pressure from low pressure to high pressure and back as pressure pulses and vacuum pulses are applied to the tissue. Certain embodiments of the invention provide means of increasing overall or basal pressure on the tissue or body part, over which protocols of high and low pressure may be superimposed. FIG. 4 illustrates the use of two sub protocols simultaneously. Such an embodiment is exemplified by a vacuum and pressure producing means producing vacuum or pressure over the body part to be treated. The aforementioned vacuum and pressure producing means is itself covered by an inflatable/deflatable balloon which provides a selectable overall or basal pressure. The basal pressure itself of course may be varied during the course of the treatment.

It is an aspect of the present invention to provide means and methods of increasing oxygen production within cells of a treated body portion as measured in terms of transcutaneous oxygen partial pressure.

It is an aspect of the present invention to provide means and methods of clearing carbon dioxide from tissues of a treated body portion as measured in terms of transcutaneous carbon dioxide partial pressure.

It is an aspect of the present invention to provide means and methods of increasing perfusion within a treated body portion.

It is an aspect of the present invention to provide means and methods of increasing NO production within a treated body portion.

It is an aspect of the present invention to provide means and methods of decreasing NO production within a treated body portion.

It is an aspect of the present invention to provide means and methods of increasing angiogenesis within a treated body portion.

It is an aspect of the present invention to provide means and methods of increasing capillary sprouting within a treated body portion.

The following example provides ample evidence of the above.

### EXAMPLE 1

### The Effect of VascuActive Device on Foot Vascular Parameters (May 8, 2009)

Abstract: A study was carried out on a healthy adult male volunteer to investigate the effect of the VascuActive device on foot perfusion and on the partial pressure of oxygen and CO₂. Blood flux, tcpO2, tcpCO2, heart rate and laser Doppler flux were measured before and during device operation, and for a period after the device was stopped. The results show an increase of tcpO2, decrease of tcpCO2, a rise in blood flux and skin temperature. These results strongly support the hypothesis that the VascuActive device enhances blood perfusion of the skin of the foot.

Methods: A healthy male volunteers (52 years old, non smoker, conducts physical exercise daily) was treated by the VascuActive device for 21 min, according to the device's protocol.

Measurements were recorded for baseline before the device was turned on (t=0 in the charts below), during its activity, and for about 6 minutes after it was turned off (the after effect). Measurements included: (*) blood flux, measured by placing the laser Doppler probe on the dorsal aspect of the foot; (*) tcpO2 and tcpCO2, measured at the foot; (*) skin temperature; and (*) heart rate.

### Results:

Blood gases measurements showed a 30% increase of tcpO₂ (Trans-cutaneous oxygen tension) during the device activation from basal values of 37 mmHg up to 48 mmHg. The level of the O₂ remained elevated after the treatment was stopped for at least 6 min (until end of measurement). The tcpCO₂ values concomitantly declined from 37 mmHg to 33-32 mmHg and this low level was maintained.

Blood flux in the foot dorsum increased from an average baseline level of 55 (arbitrary units), reaching a peak value of 90, and then, after the device was stopped, decreasing to above-baseline levels of 60-70. As explained above, these measurements were taken during vibrators non-operation periods, to avoid the artifact.

Skin temperature measurements show a continuous increase throughout the period of device activation, reaching an overall rise of 1° C. The temperature rise is characterized by three time constants: upon device activation the temperature rose about 0.4° C. in 8 min; a phase of very little rise followed, and then a steep rise of 0.6° C. to 34° C. within about 5 minutes. Temperature remained at this level for at least 6 minutes after device operation stopped.

Blood flow in the third digit, measured by an IR (infra Red) probe showed an increase of 100% in signal strength after VascuActive treatment compared to baseline level before treatment. The high level of blood flow to the digit remained for 6 minutes, until the end of data recording A similar result was recorded by the piezoelectric probe located above the ankle level.

Heart rate (HR) was calculated from blood flow data recorded during the VascuActive device operation. Heart rate values were extracted from the “Pump ON” period Heart rate declined from steady base line of 56-57 BPM to 54 BPM 8 min after start of treatment and remained so until treatment cessation. After device was stopped heart rate values increased slightly to above baseline level.

Discussion and Conclusion: Operated on a healthy, physically fit, 52 years old, non-smoking male subject the VascuActive device caused an increase of cutaneous blood flow of the foot. This increase was recorded during the treatment and after it has stopped, as reflected by the elevated tcpO₂ level and the concomitant decrease of tcpCO₂ level.

During the VascuActive treatment the blood flow in the foot dorsum, measured by laser Doppler, increased by 72% but returned to near baseline level at the end of treatment. On the other hand the blood flow in the toes, measured by IR probe, increased about 100% and remained at this level for at least 6 minutes after the treatment.

The temperature increase can be explained by vasodilatation caused by the VascuActive device.

The decrease of heart rate may be explained by a physiological response to the VascuActive treatment.

The combination of these results strongly support the assumption that the VascuActive device enhances blood perfusion and improves gas exchange balance in the foot.

It is within provision of the device to compress the body portion being treated, then during relaxation of this pressure, to expose the body portion to a fluid such as oxygen. Upon the next cycle of compression, this fluid is then forced into the tissue exposed thereto, facilitating increased rates of exchange between the tissue and the fluid thus introduced.

### EXAMPLE 2

ASSESSMENT OF THE PRELIMINARY EFFICACY OF THE VASCUACTIVE DEVICE ON FOOT PERFUSION, OXYGENATION AND PHYSICAL CAPACITY IN PATIENTS WITH PERIPHERAL VASCULAR DISEASE

Igor Rabin, MD, Arie Bass, MD

The study was conducted in the Vascular Laboratory at Assaf Harofeh Medical Center. The principal investigator in the study was Prof Arie Bass, Head of the Vascular Surgery Dept.

ClinicalTrials.gov Identifier: NCT01014377

### Introduction:

VascuActive™ is a device placed on the patient's foot for the duration of a half hour treatment session. The device implements the COMPOSITION™ technology—a proprietary, patent protected technology that combines a modified implementation of Intermittent Pressure Compression (IPC) together with oscillatory protocols—pre-programmed sequences of oscillations that span a large spectrum of frequencies, amplitudes and waveforms, originate from several generators and applied to the foot at different angles and vectors.

The objective of this trial is to study the effect of the VascuActive™ device on skin perfusion, oxygenation and walking ability of patients with peripheral vascular disease and intermittent claudication.

### Methods:

Methodology: Open, one arm, safety and efficacy, prospective trial.

Study population: 15 patients at the age range 40-70 years, arterial claudication, Fontaine II.

Treatment: Each patients was given a single half hour treatment session by the VascuActive™ device using its specifically designed oscillatory protocol

Efficacy measurements: Baseline measurements were taken for each patient just prior to the treatment and then 1 minute, 15 minutes and 30 minutes after the treatment has completed. These included measurements of skin temperature at two points in the leg and a reference point on the hand, trans-cutaneous partial pressures of O2 and CO2 of the foot, blood flux measurements using both laser Doppler and infra red fluxmetry, blood pressure and blood pressure indices. The pain-free and maximal walking distances were measured on a treadmill before the treatment and half an hour after its completion.

### Results:

Fifteen patients (14 male and 1 female) have been recruited to the study. Their mean age was 63.1±8.3 years (mean+standard deviation).

### Walking Distances

The tables below show the individual values of the Pain Free Walking Distance (PFWD) and Maximal Walking Distance (MWD) obtained for each of the patients:

Walking Distances The tables below show the individual values of the Pain Free Walking Distance (PFWD) and Maximal Walking Distance (MWD) obtained for each of the patients:

| | |
|---|---|
| PFWD (meters) | Mwd (meters) |

| Before | After | Before | After |
|---|---|---|---|
| 400 | 773 | 600 | 1115* |
| — | 489 | 300 | 691 |
| — | 96 | — | 224 |
| 120 | 235 | 210 | 499 |
| 110 | 264 | 120 | 440 |
| — | 59 | 210 | 592 |
| — | 205 | 280 | 528 |
| 60 | 528 | 100 | 645 |
| 130 | 129 | 100 | 176 |
| 113 | 118 | 220 | 411 |
| 50 | 147 | 100 | 469 |
| 125 | 240 | 300 | 1173* |
| 45 | 221 | 60 | 578 |

*in these two patients, even after 20 minutes on the treadmill the post-treatment walking test did not elicit pain that would prevent them from walking; these patients were instructed to stop by the investigators after they walked more than 1 kilometer.

The mean Pain-Free Walking Distance (PFWD) before treatment was 122±33 metersl, and it has improved to 259±54 meters after the treatment (FIG. 6). The mean change of PFWD was 155±48 meters (p=0.011)2, a relative change of 186%.

The mean Maximal Walking Distances (MWD) before and after treatment were 213±37 and 578±76 meters, respectively (FIG. 7). The change between them was 391±63 meters (p<0.001), which is a relative change of 258%.
1 Results are presented as mean±standard error, and the p value in parentheses.
2 Results are statistically significant if p<0.05. Results indicate a trend if 0.05<p<0.15. Statistically significant results are highlighted in yellow in the tables.

| | Baseline | After | Improvement | Improvement |
|---|---|---|---|---|
| | (meters) | (meters) | (meters) | (%) |
| PFWD | 122 ± 332 | 259 ± 54 | 155 ± 49 p = 0.011 | 186% ± 75 |
| | | | | p = 0.035 |
| MWD | 213 ± 37 | 578 ± 76 | 391 ± 63 p < 0.001 | 258% ± 63 |
| | | | | p < 0.01 |

### Blood Perfusion

Two laser Doppler fluxmetry probes were placed, one above the ankle and the other at the dorsum of the foot. Both peak values and amplitudes were documented. An infrared fluxmetry probe was placed over the plantar surface of the base of the first tow. FIG. 8 shows how capillary blood flow changes with treatment, as compared to the baseline measurement.

Blood flux peak measurements increased at the dorsum of foot and at the calf immediately following treatment by 41% and 43%, respectively. Amplitudes increased at the same time by 106% and 85%. Blood flow as measured by infra-red increased by 78%. Fifteen minutes later measurements decreased in varying degrees (see table below), but all remained at significantly higher than baseline levels (FIG. 9).

| | 1′ After Treatment | | 15′ After Treatment | |
|---|---|---|---|---|
| | Change (%) | P Value | Change (%) | P Value |
| Amplitude, | 106 ± 34 | 0.009 | 57 ± 34 | 0.121 |
| Calf | | | | |
| Peak, Calf | 41 ± 17 | 0.035 | 19 ± 16 | 0.28 |
| Amplitude, | 85 ± 22 | 0.002 | 72 ± 27 | 0.021 |
| foot | | | | |
| Blood, Foot | 78 ± 25 | 0.008 | 64 ± 34 | 0.081 |

### Tissue Gases

tcpO2 and tcpCO2 were measured over the dorsum of the foot. Tissue oxygen levels increased under VascuActive™ treatment by 15.8 mmHg (43%), and continued to increase steadily during the half hour afterwards, reaching a maximum increase of 28.7 mmHg (77% higher than the baseline levels). Results are statistically significant. Carbon dioxide levels decreased steadily throughout the half hour after treatment, initially only slightly, but reaching a 2.2 mmHg (5.3%) decrease after 30 minutes from treatment completion (FIG. 10).

| | | Changes (mmHg) | | |
|---|---|---|---|---|
| | Baseline | 1′ After | 15′ after | 30′ After |
| | (mmHg) | Treatment | Treatment | treatment |
| tcpO2 | 45.8 ± 4.9 | 15.8 ± 3.1 | 21 ± 4.3 (p < 0.01) | 28.7 ± 4.1 |
| | | (p < 0.01) | | (p < 0.01) |
| tcpCO2 | 39.7 ± 1.3 | 0.7 ± 1.1 | — | −2.2 ± 1.3 |
| | | (p = 0.539) | 1.8 ± 1 (p = 0.111) | (p = 0.112) |
| | | −1.2% | −4.0% | −5.3% |

### Blood Pressure Indices

Toe-Brachial Index (TBI) has increased by approximately 28% (0.14 mmHg) after the treatment has completed, and remained at the same level for the next 15 minutes. These results are statistically significant and indicate a substantial increase in foot perfusion through the small-diameter arterial vasculature of the foot. It is interesting to note the case of one of the patients, whose non-measureable toe systolic pressure before treatment (baseline TBI=0) reverted to almost normal values after the treatment: 49 mmHg at 1 minute after treatment (TBI=0.43) and 71 mmHg 15 minutes later (TBI=0.62) (FIG. 11).

Ankle-Brachial Index (ABI) has increased in a smaller magnitude of 6.6% (0.04mmHg) and returned to slightly below baseline level 15 minutes afterwards. This may indicate that blood flow through the larger diameter arteries of the leg proximal to the ankle has changed to a lesser extent.

| | | Change | |
|---|---|---|---|
| | Baseline | 1′ After Treatment | 15′ after Treatment |
| ABI | 0.69 ± 0.03 | 0.04 ± 0.02 (p = 0.026) | −0.01 ± 0.05 |
| | | 6.6% | (p = 0.79) |
| TBI | 0.51 ± 0.03 | 0.14 ± 0.02 (p < .001) | 0.14 ± 0.03 |
| | | 27.7% | (p < .001) |
| | | | 27.9% |

### Skin Temperature

Skin temperature was measured at two points on the foot—dorsum of foot and the medial aspect of the foot; and on the back of the hand, for reference. Skin temperature increased immediately after the treatment ended by 1.8°±0.5° and by 1.2°±0.3° in dorsum of foot and the ,medial aspect of the foot, respectively. After 15 minutes the temperature decrease, but to at above-baseline levels (see table below). However, when subtracting the temperature changes at the hand from those at the foot, in order to eliminate environment influences, the hand-matched temperature change was 1.1° immediately after the treatment, and it remained at a similar level for at least 15 minutes.

| | Baseline | Temperature Change (° c.) | |
|---|---|---|---|
| | (° C.) | 1′ After | 15′ After |
| Dorsum of | 29.4 ± 0.7 | 1.8 ± 0.5 | 1.5 ± 0.6 (p = 0.018) |
| Foot | | (p = 0.004) | |
| Medial | 29.1 ± 0.4 | 1.2 ± 0.3 | 0.4 ± 0.3 (p = 0.169) |
| aspect | | (p = 0.002) | |
| Difference in | | 1.1 | 1.0 |
| changes | | | |
| between | | | |
| dorsum of | | | |
| foot and | | | |
| hand | | | |

### Discussion:

The rise in laser Doppler fluxmetry and infrared fluxmetry is a direct evidence of the improved capillary blood perfusion of the foot. The concomitant rise in tissue oxygenation and carbon dioxide clearance shows the consequent net positive physiological effect of this hemodynamic change in terms of blood gases balance within the foot tissues. The rise in foot skin temperature, which was significantly higher than the changes in the hand skin temperature, provides yet another, indirect support of the conclusion that foot perfusion improved as a result of the VascuActive device treatment.

The rise in the toe-brachial index is likely the result of relaxation and decreased resistance of the small arteries of the foot, proximal to the digital artery. This hemodynamic change facilitates the rise of the capillary perfusion mentioned above. The increase in the ankle-brachial index was less than the threshold considered clinically meaningful, and further research is required to find whether it indicates a true hemodynamic effect.

The exact mechanism of action of the hemodynamic changes in the foot requires more research, but these important phenomena stand as indicators of the major hemodynamic effect of the VascuActive™ device.

Maximal Walking Distance has improved by 2.6 (p<0.01), and Pain-Free Walking Distance almost doubled, too. Since walking-related calf muscle pain is induced by the accumulation of lactate in the muscle as a result of anaerobic metabolism, the delayed appearance of pain in the post-treatment PFWD test indicates that the switch from aerobic to anaerobic metabolism occurs later than in the pre-treatment test. We assume that this is the result of improved oxygen supply to the calf muscles, due to improved blood flow, as evidenced in the foot. This is clearly a meaningful and significant clinical outcome that may have direct influence on the patient's quality of life. More research is required to learn the long term clinical outcome and the physiologic mechanisms underlying these changes.

These results clearly demonstrate that VascuActive™ device improves foot and calf muscle perfusion in a significant manner that forms the basis for changes of large magnitude in the clinical outcome, which are expressed in the pain-free and the maximal walking distances.

### Conclusion:

We conclude that VascuActive™, which implements the proprietary technology Composition™ adapted for the treatment of peripheral vascular disease of the lower limbs, increases blood perfusion to the foot, improves tissue oxygenation and carbon dioxide clearance at the foot and at the calf levels, and increases maximal and pain free walking distances. Further research is required to establish the role of this technology in the clinical setting, its position aside surgical solutions to peripheral vascular disease, and for indications such as diabetic foot, neuropathy and diabetic ulcers.

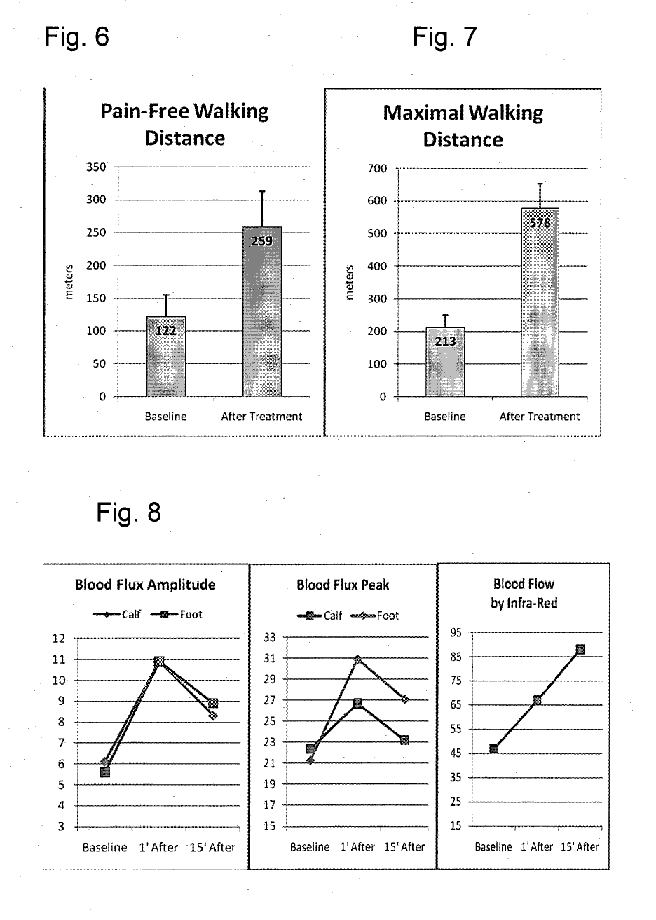

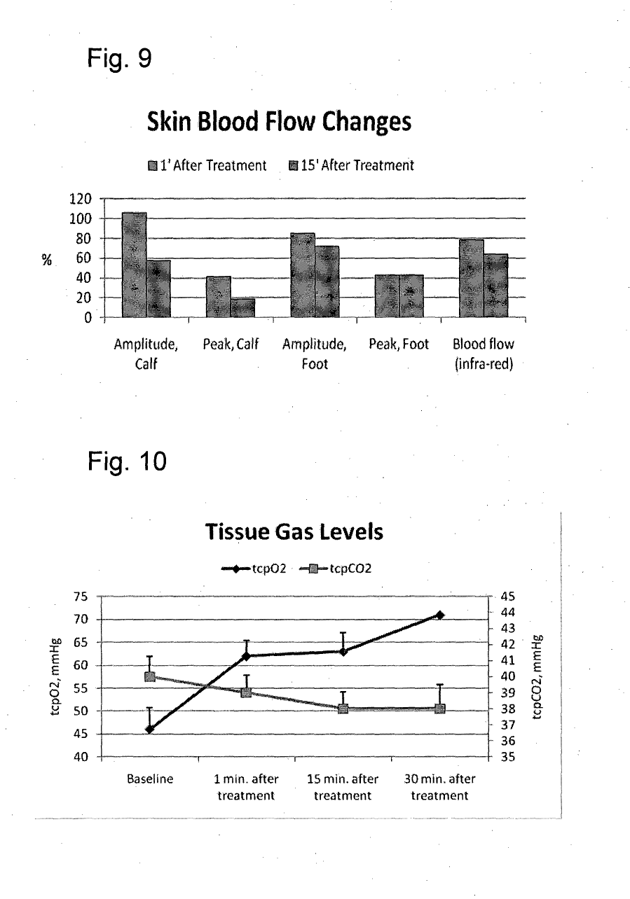

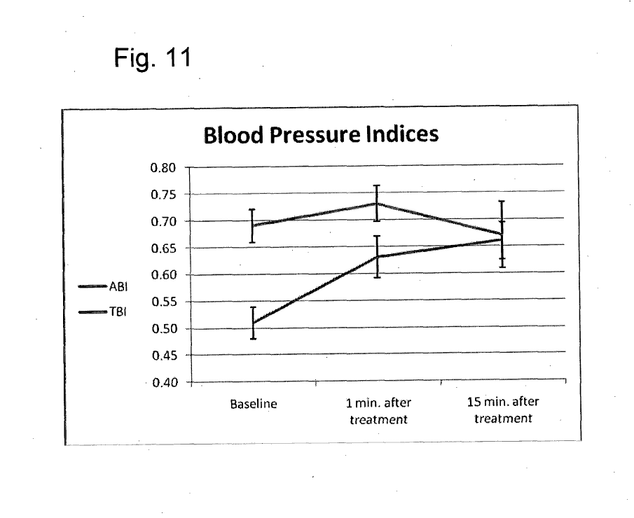

FIG. 5 herein illustrates an exemplary embodiment of the present invention in applications directed towards relief of respiratory conditions or prevention of respiratory disorders. An artificially induced vibrations device, which can be, for a non limiting example, an ADD device of patent PCT/IL2009/000962 titled DEVICE AND METHODS FOR APPLYING THERAPEUTIC PROTOCOLS TO ORGANS OF THE CARDIOPULMONARY SYSTEM (FIG. 5) (AIVD) is employed which produces artificially induced vibrations mediated through air of any of a given frequency, pressure, amplitude, volume and duration according to a protocol. The AIVD comprises:
a. a plurality of regulators module (510), each of which is adapted to define an AIV with an individual frequency, pressure amplitude, volume and duration;
b. means or mechanism module for producing the protocol (520) of said defined individual AIVs; and,
c. effector module (530) adapted to introduce said protocol of produced AIVs to impinge in or on at least at least one body portion of a patient;

Embodiments methods and protocols of the present invention are directed towards relief of respiratory conditions or prevention of respiratory disorders. An artificially induced vibrations device (AIVD) is employed which produces artificially induced vibrations mediated through air of any of a given frequency, pressure, amplitude, volume and duration according to a protocol. The AIVD comprises:
a. a plurality of regulators (510), each of which is adapted to define an AIV with an individual frequency, pressure amplitude, volume and duration;
b. means or mechanism for producing the protocol (520) of said defined individual AIVs; and,
c. effector (530) adapted to introduce said protocol of produced AIVs to impinge in or on at least at least one body portion of a patient;

FIG. 2 explains the mode of action of the abovementioned embodiment directed towards relief of respiratory conditions or prevention of respiratory disorders.

There are interactions between the applied AIVs and the natural inspirations and expirations of the patient through the patient airway.

On exhalation, air makes its way from the lungs, via the airways and out of the body On application of an air mediated AIV protocol, the patient's normal and characteristic exhalation receives therapeutic disturbances, interruptions and perturbations. The normal progress outwards encounters resistance from the applied air mediated AIV protocol and is pushed backwards towards the lungs. Depending upon the relative pressure of the air mediated AIV protocol, this result in a positive or negative pressure of the exhalation. FIG. 2a shows for example, that the air passages in the lungs are initially expanded outwards under high pressure, as incoming air encounters resistance on meeting exhaling air, creating a local pressure increase and expansion of a section of the air passage. Thus there is pressing of the expanded air passage on the surrounding tissues and blood vessels. If the protocol just provides an abrupt cessation of incoming air, there is relaxation of the airway passage. If however, the protocol provides a vacuum, or negative pressure, a tissue rebound will be induced, as illustrated. This is followed by a resumption of the exhalation outwards. Thus there is a unique protocol of wide pressure fluctuations provided by such a system, and the fig illustrates the wide range of pressure differences that can be brought about by this means and methods. This unique and predetermined pattern of events defines the frequency of operation and produces predetermined shocks, according to the predetermined protocol. The effect of rebound on the tissue will have profound effects on facilitation of blood supply, oxygenation, perfusion, CO2 and NO production and other physiologically important functions of cells and tissues. Moreover, protocols are provided to facilitate expectoration, and for expelling mucus outwards. Different characteristics are provided with protocols selected for reduction of the viscosity of mucus, for example, in CF patients, defined by specific frequencies and pressures. Yet other protocols are disclosed for gas exchange facilitation.

Reference is now made to an artificially induced vibrations device (AIVD) (FIG. 5) for producing artificially induced vibrations of any of a given frequency, pressure, amplitude, volume and duration according to a protocol; said AIVD comprising:
a. a plurality of regulators, each of which is adapted to define an AIV with an individual frequency, pressure amplitude, volume and duration. This is represented for exemplary purposes by a controller .
b. means for producing the protocol of said defined individual AIVs. This can be an air vibrator or mechanical vibrators or any other type of vibrator.
c. effector adapted to introduce said protocol of produced AIVs to impinge in or on at least at least one body portion of a patient

The aforementioned therapeutic protocol of AIVs comprises two or more different individual time resolved AIVs.

The AIVD is provided, in some embodiments, as a mattress, sheet, roll or patch, optionally provided with fixation straps or some other fixing and stabilizing device for attaching to a patient body part, bed, seat or support. The mattress, which is inflatable with fluid or gas, comprises a sheet like member and an inflatable portion and. These inflatable portions may be filled and emptied with fluid or gas by a pump according to a predetermined protocol, thereby providing a squeezing motion to the patients body portion, body part or limb or portion thereof The appropriate squeezing motion and pressure is selected according to the target tissue from a predetermined repertoire of protocols. A fluid vibrator is provided for the purpose of providing predetermined Artificially Induced Vibrations (AIVDs) in the fluid of the inflatable portion, so that the aforementioned vibrations are transferred mechanically via the sheet like member to the body portion against which the mattress is held or fixed. It is envisaged that in some embodiments of the above mentioned invention, the AIVDs will be applied mechanically to the body portion of the patient by means of vibrators.

In some embodiments of the invention the protocols are modified according to feedback information from data collected by sensors on the patient, which are transmitted to a microprocessor or central processing unit for the purpose of calculating patient related and disease related parameters and altering the administered protocol appropriately.

Reference is now made to the above mentioned artificially induced vibrations device (AIVD) wherein said AIVD is additionally provided with means for producing at least one set of predetermined humming therapeutic oscillations said humming therapeutic oscillations provided independently or contemporaneously with said series of AIVs

Reference is now made to the above mentioned artificially induced vibrations device wherein said AIVD is additionally provided with means for massaging and/or compressing at least one body portion of a patient in a predetermined manner.

Reference is now made to the above mentioned artificially induced vibrations device wherein said AIVD is adapted for external use on a patient's body.

Reference is now made to the above mentioned artificially induced vibrations device wherein said AIVD is adapted for internal use on a patient's body.

Reference is now made to the above mentioned artificially induced vibrations device wherein said AIVD is additionally provided with means for drug delivery.

Reference is now made to the above mentioned artificially induced vibrations device wherein said therapeutic protocol comprises at least one individual AIV.

Reference is now made to the above mentioned artificially induced vibrations device wherein said AIVD is adapted for administering said protocol to said patients respiratory system.

Reference is now made to the above mentioned artificially induced vibrations device wherein said AIVD is adapted for administering said protocol to said patients circulatory system

Reference is now made to the above mentioned artificially induced vibrations device wherein said AIVD is adapted for administering said protocol to said patients lymphatic system

Reference is now made to the above mentioned artificially induced vibrations device wherein said AIVD is adapted for administering said protocol to said patients cardiopulmonary system

Reference is now made to a protocol of artificially induced vibrations (AIVs) useful for improving a patients wellness; said protocol comprising sequences of different AIVs; wherein said protocol is administered to at least one body portion of the patient; further wherein said sequences are independent of said patient's natural involuntary or voluntary functions.

Reference is further made to the above mentioned protocol wherein said protocol is dependent upon said patient's natural involuntary or voluntary functions.

Reference is further made to the above mentioned protocol wherein said AIVs are transmitted to said at least one body portion of a patient by pneumatic means

Reference is further made to the above mentioned protocol wherein said AIVs are transmitted to said at least one body portion of a patient by mechanical means

Reference is further made to the above mentioned protocol wherein said AIVs are transmitted to said at least one body portion of a patient by hydraulic means

Reference is further made to the above mentioned protocol wherein said AIVs are transmitted to said at least one body portion of a patient by electrical means

Reference is further made to the above mentioned protocol wherein said AIVs are characterized by parameters selected from a group consisting of Frequency, Pressure, amplitude, Volume and Duration.

Reference is further made to the above mentioned protocol wherein said Frequency of AIVs is selected from a group consisting of about 0.5 Hz to about 5.0 Hz, about 5.0 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, any integer multiples thereof

Reference is further made to the above mentioned protocol wherein any of said selected Frequency is delivered with its corresponding overtones or multiples thereof

Reference is further made to the above mentioned protocol wherein said AIVs amplitudes are selected on the basis of clinical efficacy

Reference is further made to the above mentioned protocol wherein said volumes are selected on the basis of clinical efficacy

Reference is further made to the above mentioned protocol wherein said sequences of different AIVs is accompanied by at least one set of predetermined humming therapeutic oscillations said humming therapeutic oscillations provided independently or contemporaneously with said series of AIVs

Reference is further made to the above mentioned protocol wherein said sequences of different AIVs are accompanied by compression and/or massaging of at least one body portion of a patient in a predetermined manner

It should be noted that some embodiments of the invention are especially adapted for use on mammals, such as horses, non human primates, dogs, cats, cattle and swine. Such veterinary devices and protocols are provided as research tools. Other veterinary devices and protocols are adapted for therapeutic use. It is an aspect of the invention to provide the abovementioned device ergonomically operated by the patient or a caregiver. The device may be operated and the protocol administered independently of changes in a patients wellness, or in combination with physiological feedback from various organs, breath, respiratory gases, heart rhythm and any data derived or arising therefrom.

Some embodiments of the present invention are adapted for treating the human heart either by effecting a system such as the peripheral circulatory system and tissues close to the heart by application of the protocols on an external part of a body portion, or by an implant which is located within a body porton or cavity.

Some embodiments of the device are adapted to treat a specific organ with specific protocols designed for that organ. A useful embodiment of the invention is a balloon or balloon like member inflatable by air or liquid or other fluid and/or mechanical or electro-mechanical means for pressing the tissue or body part to effect the venous system, lymph, arteries or combinations thereof. In some embodiments, the balloon or balloon like member will be non inflatable.

In some some of the invention the protocol has the following aspects:
1. A predetermined squeezing or massaging series of actions delivered by the aforementioned balloon or balloon like balloon like member and
2. Artificially induced vibrations (AIVs) comprising predetermined sequences of different AIVs

In other embodiments of the invention the above mentioned protocol additionally comprises at least one set of predetermined humming therapeutic oscillations. These humming therapeutic oscillations are provided independently or contemporaneously with said series of AIVs

Different parts of the circulatory system may be treated; on the venous system, lymph, arteries and the pressures exerted by the device are selected approriately.

Some embodiments of the invention disclose a device for providing the abovementioned protocol protocol frequencies even without inflation.

Examples of pressures that are provided by some embodiments of the invention are:
less than—mmGH 20 for blood vessels close to the surface of the skin.
above—mmGH 20 deep veins and blood vessels.
above—mmGH 80 (diastolic pressure) cardiovascular arteries and the heart.

Higher pressures are provided for treating lymphatic oedema. For all of the above treatments, embodiments of the invention provide appropriate artificially induced vibrations of any of a given frequency, pressure, amplitude, volume and duration according to a protocol suited for the particular treatment selected.

Embodiments of the present invention include means and methods of providing protocols of AIVs with frequencies specific for:
Pain relief
Decreasing vascular resistance
Increased blood flow
Transfer of blood through arteries
Treatment of blocked blood vessels
Venous pump
Lymphatic system stimulation
NO production
Increased production of synovial fluid
Increased blood flow to promote healing of joints, tendons, muscles, sinew and cartilage
Reduction of inflammation.
Stimulation of nutrient flow to tissues such as the miniscus cartilage
Decreasing the healing time of bone fractures by stimulating bone regeneration.
Treatment of osteoporosis.

### Treatment of Nasal Congestion.

The present invention provides embodiments of the device and protocols for relief of nasal congestion. For this purpose, some embodiments of the invention include a device for delivering the aforementioned protocols of artificially induced vibrations (AIVs) to the head or face parts, or more specifically to the forehead, nose bridge, cheekbones. Patent application 60/991,772 titled SYSTEM FOR PREVENTING AND TREATING PAIN IN THE LUMBAR REGION AND ELSEWHERE IN THE HUMAN BODY PCT “A vibrating device for treating nasal congestion ” to Avni which is incorporated herein in it's entirety illustrates embodiments in a non limiting manner to which the methods and protocols of the present invention may be applied. Such aforementioned embodiments include full or partial face masks fitted especially for the purpose, with accompanying appropriate effectors. The target tissues of the protocols may be soft tissues or hard tissues. The protocols provided by these embodiments are specifically directed towards a variety of physiological and medical goals including opening of sinusitis blockage, alteration of mucus viscosity, relief and dispersal of infections, reduction of inflammation, stimulation of ciliary motion, NO generation, unblocking of blood vessels and reducing the resistance of blood vessels. Further embodiments of the present invention provide a device and protocols for drug or medication delivery through naso-phayryngeal tissues. It is herein acknowledged that drugs or medications so delivered may be specific for nasal congestion, but other drugs delivered by this method are directed to other diseases, symptoms and conditions not necessarily connected to nasal problems. An example would be drugs or medications for schizophrenia, blood pressure and pain relief.

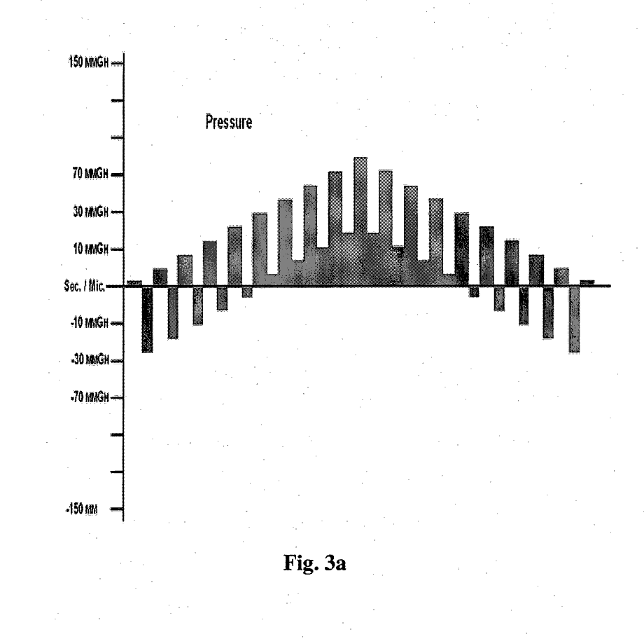

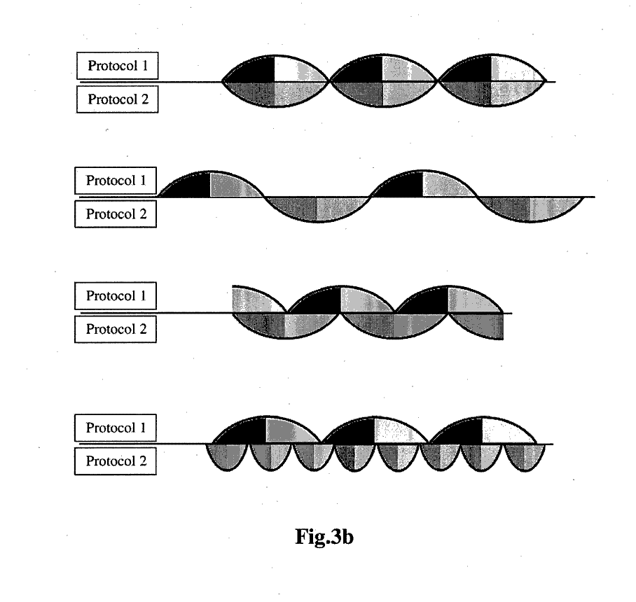

As an example of a possible protocol we refer now to FIGS. 3 and FIG. 4 Here the varied quantity, which may for instance be air pressure, voltage, position, or the like, is shown as a function of time is shown. In FIG. 3a a single value is shown while in FIG. 4 two quantities are varied simultaneously. In FIG. 4a-e further examples are given of possible protocols. These protocols may be described mathematically, for instance by expressions such as

P(t)=(Sin(ω₁t))+Bθ(Sin (ω₂t))+C,

P(t)=Floor (ASin⁻¹ (Sin (ω₁t)))+Bθ(Sin(ω₂t))+C

P=A|Sin(ω₁t)|+C

P(t)=ASin(ω₁t+σ₁)θ(Sin(ω₂t+σ₂))+C

where θ(t) is the Heaviside step function,

$$θ ( x ) = \{ \begin{matrix}1 ; & x \geq 0 \\ 0 ; & x < 0\end{matrix}$$

The protocols disclosed above can be embedded as computer programme code in CPUs, microprocessors and the like for controlling a wide range of non invasive devices, such as those described in a non limiting manner in the aforementioned patent documents herein incorporated by reference in their entirety.

1. A method of producing therapeutic oscillations useful in treating a mammalian patient comprising steps of:
a. obtaining an artificially induced vibrations device (FIG. 1) (AIVD) useful for a patients wellness for producing artificially induced vibrations of any of a given frequency, pressure, amplitude, volume and duration according to a protocol; said AIVD comprising:
a. a plurality of regulators (110), each of which is adapted to define an AIV with an individual frequency, pressure amplitude, volume and duration;
b. mechanism for producing the protocol (120) of said defined individual AIVs; and,
c. effector (130) adapted to introduce said protocol of produced AIVs to impinge in or on at least at least one body portion of a patient;
wherein said therapeutic protocol of AIVs comprises two or more different individual time resolved AIVs.

2. The method according to claim 1 wherein said protocol is dependent upon said patient's natural involuntary or voluntary functions.

3. The method according to claim 4 wherein any of said selected Frequency is deliverable with its corresponding overtones or multiples thereof.

4. The method according to claim 1 wherein said AIVD is additionally provided with means for producing at least one set of predetermined humming oscillations said humming oscillations provided independently or contemporaneously with said series of AIVs

5. The method according to claim 1 wherein said AIVD is additionally provided with means for massaging and/or compressing at least one body portion of a patient in a predetermined manner (114,115).

6. The method according to claim 1 wherein said AIVs are characterized by parameters selected from a group consisting of Frequency, Pressure, amplitude, Volume and Duration.

7. The method according to claim 4 wherein said Frequency of AIVs is selected from a group consisting of about 0.5 Hz to about 5.0 Hz, about 5.0 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, any integer multiples thereof.

8. The method according to claim 1, wherein said AIVD is adapted for administering said protocol to said patients circulatory system.

9. The method according to claim 1, wherein said AIVD is adapted for administering said protocol to at least one of said patient's lymphatic system.

10. The method according to claim 1, wherein said AIVD is adapted for administering said protocol to said patient's cardiopulmonary system.

11. A protocol of artificially induced vibrations (AIVs) useful for improving a patients wellness; said protocol comprising sequences of different AIVs; wherein said protocol is administered to at least one body portion of the patient; further wherein said sequences are independent of said patient's natural involuntary or voluntary functions.

12. The protocol according to claim 11 wherein said protocol is dependent upon said patient's natural involuntary or voluntary functions.

13. The protocol according to claim 11 wherein said AIVs are transmitted to said at least one body portion of a patient by pneumatic means.

14. The protocol according to claim 11 wherein said AIVs are transmitted to said at least one body portion of a patient by mechanical means.

15. The protocol according to claim 11 wherein said AIVs are transmitted to said at least one body portion of a patient by hydraulic means.

16. The protocol according to claim 11 wherein said AIVs are transmitted to said at least one body portion of a patient by electrical means.

17. The protocol according to claim 11 wherein said AIVs are transmitted to said at least one body portion of a patient by ultrasound means.

18. The protocol according to claim 11, wherein said AIVs are characterized by parameters selected from a group consisting of Frequency, Pressure, Amplitude, Volume and Duration.

19. The protocol according to claim 18, wherein said Frequency of AIVs is selected from a group consisting of about 0.5 Hz to about 5.0 Hz, about 5.0 Hz to about 10 Hz, about 10 Hz to about 20 Hz, about 20 Hz to about 30 Hz, about 30 Hz to about 40 Hz, about 40 Hz to about 50 Hz, about 50 Hz to about 60 Hz, about 60 Hz to about 70 Hz, about 70 Hz to about 80 Hz, about 80 Hz to about 90 Hz, about 90 Hz to about 100 Hz, any integer multiples thereof.

20. The protocols according to claim 19 wherein any of said selected Frequency is delivered with its corresponding overtones or multiples thereof.

21. The protocols according to claim 19 wherein said AIVs amplitudes are selected on the basis of clinical efficacy.

22. The protocols according to claim 19 wherein said volumes are selected on the basis of clinical efficacy.

23. The protocol according to claim 18 wherein said sequences of different AIVs is accompanied by at least one set of predetermined humming therapeutic oscillations said humming therapeutic oscillations provided independently or contemporaneously with said series of AIVs.

24. The protocol according to claim 18 wherein said sequences of different AIVs are accompanied by compression and/or massaging of at least one body portion of a patient in a predetermined manner.

25. The protocol according to claim 46, wherein said vibrations are modulated according to a function selected from the group consisting of:
P(t)=ASin⁻¹ (Sin(ω₁t))+Bθ(Sin(ω₂t))+C,
P(t)=Floor(ASin⁻¹ (Sin(ω₁t)))+Bθ(Sin(ω₂t))+C,
P(t)=A|Sin (ω₁t)|+C ,
P(t)=ASin (ω₁t+σ₁)θ(Sin(ω₂t+σ₂))+C.

26. The protocol according to claim 25 where: 0.01 mm≦A≦10 mm, 0.01 mm≦B≦10 mm, 0.01 mm≦C≦10 mm, 0.01 Hz≦ω₁≦100 Hz, 0.01 Hz≦ω₂≦100 Hz.

27. The method of claim 1, wherein said artificially induced vibrations (AIVs) are administered as a protocol comprising sequences of different AIVs; said protocol is administered to at least one body portion of the patient; wherein said AIVs are in the form of pulsating negative pressures, or pulsating negative and positive pressures wherein said protocol of AIVs comprises one, two or more different individual time resolved AIVs.

28. The method of claim 27 wherein said pulsating negative pressures, or pulsating negative and positive pressures are produced in the respiratory system.

29. The method of claim 27 wherein said pulsating negative pressures, or pulsating negative and positive pressures are produced in the respiratory system of a mammalian patient.

30. The method of claim 27 wherein said pulsating negative pressures, or pulsating negative and positive pressures are produced in the circulatory system of a mammalian patient

31. The method of claim 27 wherein said pulsating negative pressures, or pulsating negative and positive pressures are produced in the cardiovascular system of a mammalian patient.
